(19) United States
(12) Patent Application Publication
(10) Pub. No.: US 2015/0072017 A1
(43) Pub. Date: Mar. 12, 2015

(54) CARRIER MATERIALS FOR PROTEIN DELIVERY

(71) Applicant: Olympus Biotech Corporation, Hopkington, MA (US)

(72) Inventors: Hyun Kim, Weston, MA (US); Shinichi Torii, Marlborough, MA (US); Margaret M. Worden, Hanover, MA (US); Tetsuro Ogawa, Tokyo (JP); Nozomi Komatsu, Tokyo (JP)

(73) Assignee: Olympus Biotech Corporation, Hopkington, MA (US)

(21) Appl. No.: 14/021,201

(22) Filed: Sep. 9, 2013

(51) Int. Cl. A61L 27/54; A61L 27/24; A61L 27/56; A61L 27/18

(52) U.S. Cl. CPC A61L 27/54; A61L 27/18; A61L 27/24; A61L 27/56; A61L 2430/02; A61L 2400/06; A61L 2300/414
USPC 424/499; 514/769; 514/8.8

(57) ABSTRACT

Osteogenic implants, carriers and concentrates are described, along with methods of making and using the same. The implants include a carrier and, optionally, an osteoinductive agent. The carrier includes a mineral component, a binder and, optionally, a collagen additive, while the osteoinductive agent may be a protein such as a bone morphogenetic protein.

### TECHNICAL FIELD

The invention relates to protein carriers for use in medical applications, and specifically for carriers for delivering proteins to musculoskeletal tissues.

### BACKGROUND

Historically, patients with severe orthopedic injuries such as fractures, traumatic injuries, or skeletal defects required bone grafts in order to rebuild damaged musculoskeletal structures. Bone grafts generally come from one of two sources: tissues harvested from one healthy region of a patient to be used to treat an injured part of that patient (termed “autografts”), and tissues harvested from another individual, typically a cadaveric donor (termed “allografts”). Grafted tissue may stimulate the growth of injured bone by a variety of mechanisms, including osteoinduction (driving the proliferation of osteoprogenitor cells that form bone) and osteoconduction (acting as a scaffold for the deposition of new bone material). Autograft tissue exhibits both osteoinductive and osteoconductive properties, and has been used for many years in a variety of orthopedic procedures including trauma, nonunion, spine fusion, foot/ankle fusion, or other bone defects, but autograft tissue is generally available only in limited quantities, and patients may suffer complications associated with the harvesting procedure. Cadaveric allograft tissue is often used as an alternative to autografts, but allograft supply is still restricted, and allograft material may have limited osteoinductivity. There may also be a risk of transfer of disease by allograft tissue from donor to recipient, which requires screening of allograft tissue for pathogens.

In spite of their usefulness in the clinic, the drawbacks of autograft and allograft materials highlight the need for alternative synthetic products for the effective treatment of orthopedic injuries. Synthetic calcium phosphate-based materials have been employed clinically as bone void fillers for several decades. Such materials generally do not suffer from the same issues associated with graft tissue, and have the potential to become viable alternatives to graft tissue if their osteoinductive properties can be improved. To that end, some calcium-based bone void fillers have been investigated as potential carriers for osteoinductive molecules, particularly bone morphogenetic proteins (BMPs).

A number of commercially available bone graft substitutes that incorporate the primary structural components of bone (e.g. collagen and calcium based compounds) have been developed and are widely used. These substitutes, including ceramics, hydroxyapatites and tricalciumphosphates, function primarily by physical, osteoconductive means, facilitating cellular attachment and migration from surrounding bone. They provide some graft function without donor site morbidity, have acceptable biocompatibility and have limited risk of disease transmission. These materials are most often provided in the form of small, porous granules that can be packed to fill the wide variety of sizes and shapes of bony defects encountered. Recently, several products have been introduced that combine granular calcium phosphates with collagen or other materials that act to bind the granules together into certain shapes (e.g. strips, blocks) to improve delivery and retention of the granules at the graft site. Such products have been demonstrated to be osteoconductive, providing a scaffold for cell attachment and supporting formation of osseous tissue across joints in fusions.

While the osteoconductive properties of calcium phosphate materials are widely accepted, calcium phosphates have historically not been considered to possess osteoinductive potential. However, adding bone morphogenetic proteins (BMPs) to osteoconductive scaffolds may result in improved osteoinductivity. Combinations of BMPs with osteoconductive scaffolds or carriers may have the advantage of outstanding osteoinductivity and provide patients with an alternative choice to other bone grafting procedures. Several existing combination products utilizing delivery of BMPs with carriers are commercially available, including OP-1 Implant™ (e.g., Osigraft™, available in Europe) and OP-1 Putty™ (e.g., Opgenra™, available in Europe), both commercialized by Olympus Biotech, Hopkinton Mass., as well as Infuse® bone graft, commercialized by Medtronic Inc., Minneapolis, Minn. The OP-1 Implant™ includes Eptotermin Alfa (rhBMP-7/OP-1) provided with collagen granules and indicated for long bone nonunions. The OP-1 Putty™ is similar to the OP-1 Implant™ product with the addition of carboxymethylcellulose as a putty additive and is indicated for posterolateral spine fusion. Infuse is rhBMP-2 provided with a collagen sponge for a variety of indications including open tibia fracture, interbody spine fusion, and dental applications. In addition to these commercial products, there are a number of publications and intellectual property involving the composition and use of various carriers, scaffolds, or delivery systems for a variety of BMPs.

Synthetic calcium-based osteoinductive products may have significant advantages over currently-used human-derived graft materials if they can supplant allograft and autograft tissues as the materials of choice for repairing severe orthopedic conditions with improved safety and efficacy profiles.

### SUMMARY OF THE INVENTION

Embodiments of the current invention address the ongoing need in the art for synthetic osteoinductive products having improved safety and efficacy by providing osteogenic implants and protein carriers that include a calcium-based matrix with an optimized pore structure and a dissolvable, reverse thermosensitive binder such as a poloxamer, as well as methods of making and using the same.

In one aspect, the invention relates to an implant comprising β-tricalcium phosphate particles, a binder, and an osteogenic agent. In various embodiments, the β-tricalcium phosphate particles have macropores and micropores, and a porosity of between about 50% and about 90%. The tricalcium phosphate particles optionally have a diameter of between about 0.1 mm and 3.0 mm. The binder is, in some embodiments, a poloxamer such as poloxamer 407. The osteogenic agent is, in various embodiments, a bone morphogenetic protein, for example recombinant human BMP-7 (rhBMP7). The implant comprises, variously, a therapeutic material in the form of bone putty, a bone paste, a slurry, or a solid body sized to be placed into a patient, for example by injection.

In another aspect, the invention relates to a concentrate dilutable to form a therapeutic material, which can take the form of a bone paste, a bone putty, a slurry or a solid body. The concentrate includes between about 50% and about 80% by mass of β-tricalcium phosphate particles and between about 20% and about 50% by mass of a binder which may be a poloxamer such as poloxamer 407, and optionally includes a collagen, which may be atelocollagen or native collagen and/or may be in particulate form. The β-tricalcium phosphate particles may have a diameter of about 0.1-3.0 mm, and may have a porosity between about 50% and about 90%. The particles may also include a macropore and a micropore. In some embodiments, the particles include hydroxyapatite.

In yet another aspect, the invention relates to a kit for treating a patient that includes a concentrate comprising β-tricalcium phosphate particles and a binder which may be a poloxamer such as poloxamer 407, lyophilized bone morphogenetic protein, and an aqueous diluent. The kit may also include instructions for performing a method that includes the steps of adding the diluent to the lyophilized bone morphogenetic protein to form a bone morphogenetic protein solution and mixing a quantity of the protein solution with a quantity of the concentrate to form a therapeutic material in the form of a bone paste, a bone putty, a slurry or a solid body. The therapeutic material may be flowable in some instances, and the method may include flowing the therapeutic material into a body of a patient, while in other instances the therapeutic material may be moldable, and the method may include molding the therapeutic material to fit, at least partially, into a void within a bone of a patient.

And in yet another aspect, the invention relates to a method of treating a patient that includes adding a quantity of a bone morphogenetic protein solution to a concentrate comprising β-tricalcium phosphate particles and a poloxamer such as poloxamer 407 to form a therapeutic material in the form of a bone putty or a bone paste, and placing the therapeutic material in the body of a patient.

### DRAWINGS

In the drawings, like reference characters refer to like features throughout the different views. The drawings are not necessarily to scale, with emphasis being placed on illustration of the principles of the invention.

FIG. 1 is a photomicrograph illustrating the microporous structure of calcium phosphate materials used in various embodiments of the invention.

FIG. 2 is a photomicrograph illustrating the macroporous structure of calcium phosphate materials used in various embodiments of the invention.

FIG. 3 depicts the results of an in vitro assay for release of rhBMP-7 from TCP/poloxamer carriers of the invention.

FIG. 4 depicts the results of an in vivo radiographic scoring for bone formation in a rabbit long bone segmental defect model treated with implants comprising rhBMP-7 and TCP/poloxamer carriers of the invention.

FIG. 5 depicts the results of an in vivo torsional bone strength assessment in a rabbit long bone segmental defect model treated with implants comprising rhBMP-7 and TCP/poloxamer carriers of the invention.

FIG. 6 depicts the results of ADA assays for rhBMP-7 following implantation of implants of the invention in a rabbit femoral condyle model.

FIG. 7 depicts the results of ADA assays for rhBMP-7 following implantation of implants of the invention in a rabbit segmental defect model.

### DETAILED DESCRIPTION OF THE PREFERRED EMBODIMENTS

### Osteogenic Implants:

An osteogenic implant of the invention includes a carrier and an osteogenic protein. The carrier is, in various embodiments, a biodegradable material that is fluid but which may include suspended or partially solubilized solids (e.g. a slurry, paste or cement), a plastic, moldable solid (e.g. a putty), or a rigid solid body (e.g. a solid particle or rod). The carrier has both a mineral component and an excipient or binder, and optionally includes a collagen additive that may additionally improve the handling characteristics or biocompatibility, as discussed in more detail below.

The mineral component of the carrier is generally comprised of calcium phosphate and is preferably tricalcium phosphate (TCP), and more specifically β-TCP. In some cases, the mineral component is biphasic, consisting of β-TCP and hydroxyapatite. Generally, the mineral component is in the form of granules between the size range of 0.1-3.0 mm, preferably 0.2-2.0 mm, more preferably 0.25-1.5 mm, and most preferably 0.25-0.5 mm. The preferred embodiment of the mineral component is β-TCP in the form of porous granules having a porosity of ≧50%. The pore structure of each granule preferably includes a combination of interconnected macropores (≧100 μm and preferably 100-400 μm) and micropores (≦10 μm and preferably about 1 μm). Each granule contains micropores, macropores, or, most preferably, a combination of both. The porosity of TCP is ≧50%, preferably 60-90%, more preferably 60-75%, and most preferably about 75%. The combination and interconnection of the micropores and macropores within the particle increases the surface area available for cell attachment and osteogenic protein adsorption. These porosity characteristics result in granules that degrade on a time-scale that is aligned with new bone formation: if a matrix degrades too rapidly, it creates voids in the graft area that inhibit ingrowth of new bone, while degradation that is too slow prevents infilling of new bone material into the space occupied by the graft material, also inhibiting new bone formation. The mineral components of osteogenic implants of the current invention are characterized by an optimized porosity, which helps insure that residual matrix material is present when it needs to be without interfering with new bone formation.

The binder is a synthetic or natural polymer whose function is to serve as a temporary glue that holds the mineral granules together and, optionally, to act as an excipient for a therapeutic agent. The choice of binder and its concentration in the carrier influences the physical characteristics of the osteogenic implant—for instance, the use of a binder that is plastic and deformable can help contribute to plasticity and deformability in the final implant (e.g. a putty). The binder also allows the implant to be extrudable through a cannula or molded into shape manually by hand. Any suitable synthetic or natural materials can be used in other embodiments of the invention, including without limitation pluronics, polyvinylpyrrolidone, cellulose, methylcellulose, carboxymethylcellulose, hyroxypropylmethylcellulose, alginate, chitosan, xanthan gum, collagen, fibrin, elastin, proteins, proteoglycans, hyaluronic acid, polyesters, polylactide, polyglycolide, polycaprolactone, polyvinyl alcohol, polyethylene glycols, and other polymers. In preferred embodiments of the present invention, however, the binder is a poloxamer such as poloxamer 124 (Pluronic® L44), poloxamer 188 (Pluronic® F68), poloxamer 237 (Pluronic® F87), poloxamer 338 (Pluronic® F108), and most preferably poloxamer 407 (Pluronic® F-127 (BASF, Ludwigshafen am Rhein, Germany)).

Poloxamers are water soluble polypropylene oxide polyethylene oxide (PPO/PEO) triblock co-polymers which undergo a reversible sol-gel phase transition at a predetermined critical temperature, such that a poloxamer solution will have a first, relatively lower viscosity below the critical temperature and a second, relatively higher viscosity above the critical temperature. Poloxamers used in the present invention generally have a molecular weight range of about 6,840 to about 17,400. Poloxamer 407 is a PPO/PEO triblock copolymer with an average molecular weight of 12,600 and a molecular weight range of about 9,840 to about 14,600. Poloxamer 407 includes a central PPG block of about 56 repeats flanked on both sides by PEG blocks of about 101 repeats each. The temperature sensitivity, surfactant and stabilizing properties of poloxamers generally, and of poloxamer 407 in particular contribute to their suitability as binders in various embodiments of the invention. Poloxamer 407 also includes the advantage of a relatively high viscosity of about 3,100 cps. At lower temperatures, which may exist when an implant of the invention is first delivered to a patient, the implant has a relatively low viscosity or high degree of deformability, as the case may be, facilitating its delivery and the tailoring of its shape to a site of implantation. However, as the implant is warmed by the body, its viscosity will increase, or its deformability will decrease, and the implant will become more rigid and, accordingly, will be less likely to become displaced. Additionally, in carriers of the invention, the binder (e.g., poloxamer) temporarily binds together TCP granules during the preparation, implantation, and initial stages of repair but later solubilizes in vivo thereby forming additional porosity (in addition to micro- and macro-pores within TCP granules) for the host cells to migrate into the scaffold and fill in between the TCP granules, and render the rhBMP-7 bioavailable to the cells for initiation of the bone formation cascade, as discussed in more detail below.

Preferred carrier embodiments have β-TCP compositional ranges of 20-90 wt %, and preferably 50-80 wt %, and have binder compositional ranges of 10-80 wt %, and preferably 20-50%. The preferred composition for β-TCP to binder is 67% to 33% (w/w). Putties and pastes of the invention also include an aqueous component, and the range of wetting ratios to prepare a moldable putty is 0.5-0.7 mL of liquid per 1 g of solid (L/S ratio of 0.5-0.7). Preferably, the L/S ratio is 0.55-0.65, and most preferably the L/S ratio is 0.6 (e.g., 0.6 mL reconstituted rhBMP-7 to 1 g dry TCP/poloxamer mixture). Putties and pastes of the invention can optionally include a collagen additive.

The addition of atelocollagen (protease treated collagen) or telocollagen (native collagen) to carriers of the invention may additionally improve their handling characteristics and bone repair by yielding a slightly drier and more cohesive moldable putty while offering the potential for improved host cell recognition and attachment. Collagens for use in carriers of the invention can be bone, dermal, or tendon derived Type I collagen or other types of collagens (Type II, III, IX, X, and others) of natural or synthetic origin. The preferred collagen is Type I atelocollagen. Atelocollagen is a pepsin-treated collagen to remove the telopeptides which as a result improves acid solubility and reduces immunogenicity. Atelocollagen can be further cross-linked by chemical or heat cross-linking.

The active component of an osteogenic implant according to the invention is generally an osteogenic protein, which is typically one or more bone morphogenetic proteins (BMPs) or derivatives thereof, and/or other growth factors known to promote bone formation such as growth differentiation factors (GDFs). BMPs for use in osteogenic implants of the invention include BMP-2, BMP-4, BMP-5, BMP-6, BMP-7, BMP-9, BMP-11, BMP-12, BMP-13, and their peptides, analogues, variants, and combinations thereof. In preferred embodiments of the invention, BMP-7 is used, most preferably recombinant human BMP-7 (rhBMP-7).

Osteogenic implants of the invention may take a variety of forms, but will preferably be one of a moldable putty and an injectable/extrudable paste, both of which are intended for use as an implanted or injected product either through open reduction surgery or percutaneous injection into skeletal sites that may require repair, regeneration, augmentation, grafting in a variety of orthopedic indications including long bone nonunions, fracture repair, spine fusion, foot/ankle fusion, vertebral compression fractures, local bone augmentation, oral/maxillofacial surgery, osteoporosis, osteolysis, and other bone defect/injury repair.

Osteogenic implants according to the invention—which contain the carriers and osteogenic proteins described above—exhibit superior performance and safety relative to currently available products. One advantage of these implants is the temporary binding effect of the binder component of the β-TCP/poloxamer carrier, which allows an implant of the invention to cohere (e.g. to be a solid body, plastic body, paste or slurry) prior to implantation; once implanted, the implant disintegrates over time via dissolution of the binder component, leaving only the β-TCP granules at the site of implantation. As the binder disintegrates, it contributes to the formation of inter-granular porosity among the β-TCP granules, which in turn allows cellular ingrowth of host pluripotent cells into the site of implantation. This cellular migration, in turn, improves the bioavailability of the rhBMP-7, and insofar as the ingrowing cells are undifferentiated, their exposure to rhBMP-7 results in the initiation of the bone-formation cascade. The disintegration of the cohesive putty carrier into granular form would be expected to initiate within hours and continue for several days in vivo, which is critical for the cells to penetrate the carrier matrix. In the absence of this disintegration, there is a risk of bone shell encapsulation over the implanted carrier due to limited penetration of cells into the carrier matrix.

In addition to the creation of inter-granular voids (porosity between granules as a result of disintegration of the binder), the pre-existing porosity within the β-TCP granules (in the form of micropores and/or macropores) increases the surface area available for cell attachment and rhBMP-7 adsorption. The inventors have observed, in multiple orthotopic animal models (as discussed in greater detail below), unexpectedly enhanced bone formation, in terms of accelerated healing rate and the degree of healing, after application of implants comprising β-TCP/poloxamer carriers and rhBMP-7 according to the invention. The superior radiographic healing results encountered with the carriers in the current invention over autograft were unexpected. Also surprising was a decrease in inflammation and immunogenicity against rhBMP-7 in experimental animals. These findings are detailed in the examples below.

### EXAMPLES

The principles of the invention in its various embodiments are illustrated by the following non-limiting examples:

### Example 1

### Carrier Composition and Formulation Examples and Ranges

As discussed above, carriers of the invention include a mineral component which is generally calcium phosphate, preferably tricalcium phosphate (TCP), and most preferably β-TCP (Ca₃(PO₄)₂). The mineral component can be biphasic in some embodiments, consisting of β-TCP and hydroxyapatite. The mineral component is in the form of granules within the size range of 0.1-3.0 mm, preferably 0.2-2.0 mm, more preferably 0.25-1.5 mm, and most preferably 0.25-0.5 mm.

Carriers of the invention also include a binder, a synthetic or natural polymer which functions as a temporary glue that holds the mineral granules together, and optionally acts as an excipient for the active ingredient. The binder provides handling improvements over mineral component alone by permitting the carrier to take the form of a shapeable, moldable putty and/or to be extrudable through a cannula or molded into shape manually by hand.

One preferred carrier embodiment, TCP/poloxamer, is a synthetic moldable material comprised of β-TCP granules (0.25-0.5 mm range; 75% porosity including micro- and macro-pores) and a poloxamer binder (most preferably poloxamer 407) at a ratio of 2:1 (weight:weight) TCP:poloxamer (or 67:33 w/w %). While a variety of TCP formulations are currently approved for patient use, the inventors have found that OSferion® β-tricalcium phosphate (commercialized by Arthrex, Inc., Naples, Fla.) is suitable for use in carrier preparations of the present invention, although other β-TCP products having similar specifications and an acceptable pore structure can be used.

### Example 2

### Collagen Enhanced Carrier Formulations

The addition of collagen to synthetic moldable TCP/poloxamer carriers may improve their osteoconductivity and handling. Generally, collagens from two sources will be used in carriers of the invention: (i) demineralized bone matrix derived Type I telocollagen of bovine origin, and (ii) bovine dermal derived Type I atelocollagen. In preferred embodiments, Type I atelocollagen is used. Type I atelocollagen is a pepsin-treated collagen which lacks telopeptides and exhibits increased acid solubility and reduced immunogenicity compared to telocollagen. Atelocollagen can be further cross-linked by chemical or heat cross-linking to prolong degradation time.

Putties of the invention preferably include β-TCP for the mineral component, poloxamer as the binder component, and atelocollagen as the collagen component. Putties comprising atelocollagen generally include 10-80% TCP, 2-25% atelocollagen, and 10-50% poloxamer (all wt %), respectively. One exemplary carrier is composed of TCP/atelocollagen/poloxamer at 53/5/42 wt %. The range of wetting ratios to prepare a TCP/atelocollagen/poloxamer moldable putty is 0.43-0.63 mL of liquid per 1 g of solid. Preferably, the ratio is 0.48-0.58 mL liquid/g solid, and most preferably the L/S ratio is 0.53 (i.e., 0.53 mL reconstituted rhBMP-7 in solution to 1 g dry TCP/atelocollagen/poloxamer mixture).

Putties comprising telocollagen generally include 10-80% TCP, 5-80% telocollagen, and 10-50% poloxamer (all wt %), respectively. The preferred compositional range for TCP/telocollagen/poloxamer is 50/25/25 wt %. The range of wetting ratios to prepare the TCP/telocollagen/poloxamer moldable putty is 0.8-1.0 mL of liquid per 1 g of solid (L/S ratio of 0.8-1.0). Preferably, the L/S ratio is 0.85-0.95, and most preferably the L/S ratio is 0.9 (i.e., 0.9 mL reconstituted rhBMP-7 to 1 g dry TCP/telocollagen/poloxamer mixture).

### Example 3

### Pore Size and Porosity Ranges of β-TCP Granules and β-TCP/Poloxamer Moldable Putty

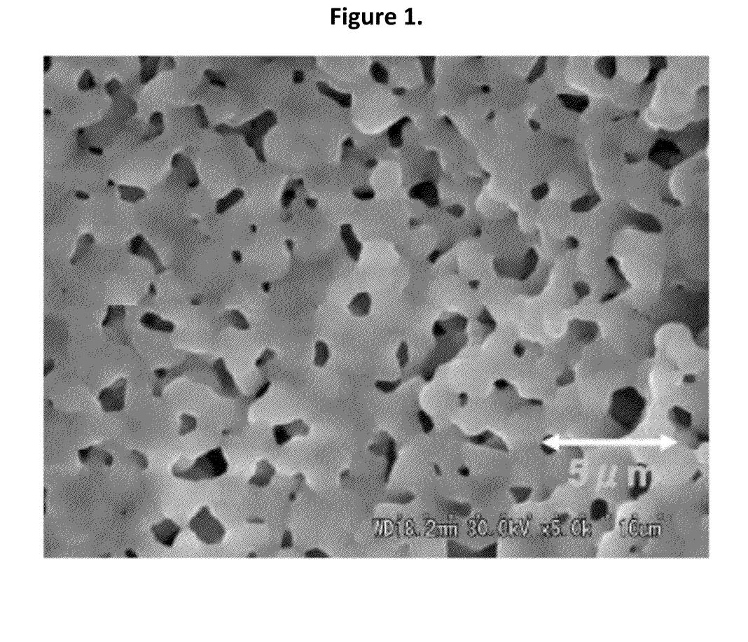

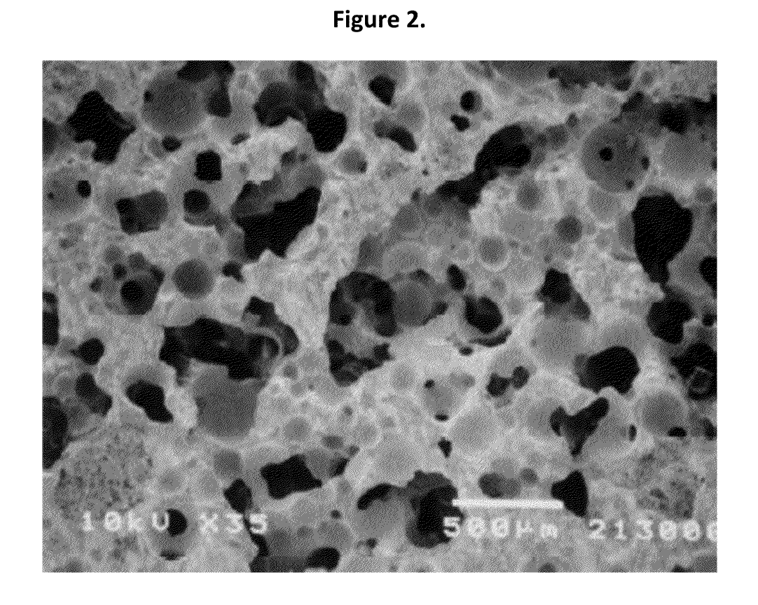

The preferred β-TCP material for use in carriers of the invention comprises porous granules, exhibiting intra-granular porosity of ≧50% distributed within each granule as a combination of interconnected macropores (≧100 um an preferably 100-400 um; FIG. 2) and micropores (10 um and preferably about 1 um; FIG. 1). The porosity of the TCP is ≧50%, preferably 60-90%, more preferably 60-75%, and most preferably 75%. The combination of interconnected micropores and macropores increases the surface area available for cell attachment and osteogenic protein adsorption. The relatively high porosity of the granules also promotes biodegradation on a timescale aligned with new bone formation such that the residual matrix does not interfere with new bone formation, as described above.

Another important feature of carriers and implants of current invention is their high-degree of inter-granular porosity. Inter-granular porosity refers to the degree to which pores form between individual TCP granules as the carrier disintegrates into TCP granules by way of solubilization or dissolution of the binder. The development of inter-granular pores enables robust cellular ingrowth into the implanted matrix filling the voids created between the individual TCP granules. This cellular infiltration, in turn, increases the bioavailability of osteoinductive agents adsorbed to the TCP granules. The preferred inter-granular pore size range is ≧100 um, and more preferably 100-500 um, similar to pore sizes of cancellous bone of approximately 200-400 um.

### Example 4

### In Vitro Binding and Release of rhBMP-7

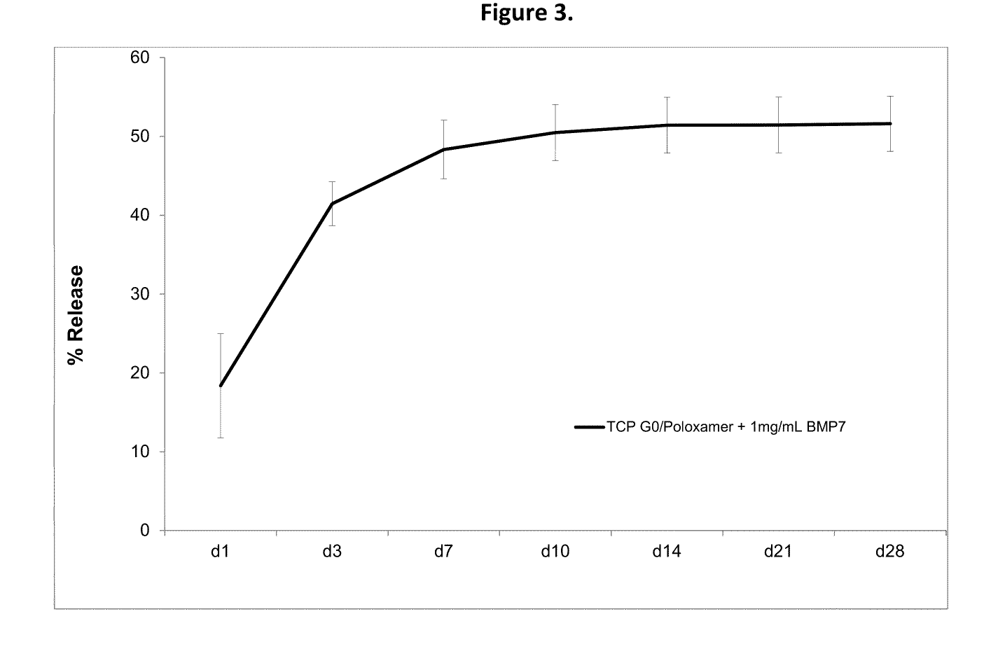

While not wishing to be bound to any theory, it is believed that rhBMP-7 tends to associate strongly with TCP granules in compositions of the invention due to electrostatic interactions across the relatively large surface areas generated by the pore structures described above. Preliminary binding studies on TCP granules of different types suggested that carriers of the invention have increased rhBMP-7 binding capacity likely due to the larger surface area. A preliminary in vitro release study was conducted on implants comprising TCP/poloxamer and 1 mg/mL rhBMP-7 (i.e., 1 mg rhBMP-7 per 1 mL of carrier). Approximately 50 mg of carrier with rhBMP-7 was submerged in 1 mL of PBS at pH 7.4 at 37° C. in a shaker water bath at 100 rpm over 28 days. At each time point, the supernatant was drawn out and replaced with fresh PBS. The samples were analyzed for rhBMP-7 content using enzyme-linked immunosorbent assay (ELISA). A two-phase release was observed, with an initial burst during the first 3 days followed by sustained release up to 2 weeks (FIG. 3).

While not wishing to be bound to any theory, the inventors believe that a fraction of the rhBMP-7 within the implant remains bound to TCP granules throughout its use; this fraction accounts for a difference between the amount of rhBMP-7 loaded into the implant and the cumulative release of rhBMP-7 from the implant. This fraction would be expected to become bioavailable via contact with infiltrating cells as the binder dissolves or disintegrates to create intra-granular pores within the TCP matrix.

### Example 5

### Carrier Evaluation in Rat Ectopic Bone Formation Model

Initial carrier screening in vivo was performed in a rat ectopic bone formation model in which a number of different carriers, including carriers according to the invention, were evaluated in combination with rhBMP-7. In particular, TCP/poloxamer carriers were evaluated and compared with currently marketed implants that deliver BMPs (i.e., OP-1 Implant, Osigraft). Tested implants of the invention were prepared to comprise 1 mg rhBMP-7 per mL of carrier volume (1 mg/mL), similar to the final concentration of rhBMP-7 in OP-1 Implant (1 mg/mL). Three weeks after implantation of each carrier (0.3 mL carrier volume; n=3 rats [6 implants] per group) into the dorsal lumbar region in the subcutaneous site of the rat, explants were evaluated for bone formation and inflammation using microscopic evaluation. The mean bone scores were similar (up to 25% new bone) between TCP/poloxamer implants of the invention and currently marketed implants. In addition, local inflammatory response was mild to moderate in the TCP/poloxamer carrier and comparable to OP-1 Implant.

### Example 6

### Bone Formation Efficacy in Rabbit Femoral Condyle Bone Defect Model

Several leading carrier candidates including TCP/poloxamer carriers of the present invention were evaluated in a more clinically relevant rabbit femoral condyle metaphyseal bone defect model. Bilateral 5 mm diameter by 8 mm deep cylindrical defects were created in the lateral femoral condyle. Each carrier alone or with 1.0 mg/mL rhBMP-7 (n=4 implants per group) was implanted and compared with OP-1 Implant and empty defect after 4 weeks in-life. The primary outcome assessment was bone formation and, secondarily, the level of inflammation by microscopic evaluation. Additionally, anti-drug antibody (ADA) response against rhBMP-7 in serum samples was preliminarily evaluated at baseline and weekly thereafter using an in-house ELISA based direct assay.

Microscopic analysis of the defect area revealed that the amount of new bone formation was greater in the TCP/poloxamer compositions of the current invention (50-75% new bone area) containing rhBMP-7 compared to other carriers (25-50% new bone area), whereas bone formation was comparable between other carriers containing rhBMP-7 and OP-1 Implant. Furthermore, in an additional group, bone formation of rhBMP-7 combined with TCP alone without poloxamer was comparable to rhBMP-7 combined with TCP/poloxamer, suggesting no negative impact of poloxamer on TCP or rhBMP-7.

These results indicate that compositions and carriers of the invention have osteoconductive activity that is complementary to osteoinductive effects of rhBMP-7, and that the addition of poloxamer to TCP does not appear to interfere with bone formation. TCP/poloxamer appears to induce little to no inflammation within the implanted site, in contrast to telocollagen-containing carriers, which may prolong the local inflammatory response, delay bone formation, and promote an antibody response to rhBMP-7.

### Example 7

### Bone Formation Efficacy in Rabbit Long Bone Segmental Defect Model

The purpose of this study was to evaluate the rhBMP-7 dose response in a TCP/poloxamer carrier, and to determine the relative benefit of the addition of 2 different sources of collagen to the TCP/poloxamer carrier. TCP/poloxamer, along with TCP/poloxamer with either telocollagen (same collagen used in OP-1 Implant) or atelocollagen (different collagen from OP-1 Implant), were evaluated in the rabbit radius 20 mm segmental defect model as a dose ranging study to determine the minimally efficacious rhBMP-7 dose and a therapeutic range. This nonunion/delayed healing model was chosen because of its clinical relevance to the long bone nonunion indication. Implants containing 0, 0.025, 0.1, and 1.0 mg/mL rhBMP-7 were implanted bilaterally in 6 rabbits (n=12 total per group) for 6 weeks and evaluated for radiographic healing (at post-op and weeks 2, 4, 5, 6) as a primary measure with secondary histomorphometry and histopathology measures. Radiographic healing was scored on a 0 to 6 scale, with 6 being complete healing (Cook et al., Clin Orthop Related Res 301: 302-312, 1994). OP-1 Implant and autograft served as positive controls, while empty defect served as a negative control. Mechanical testing (torsional strength) was performed on additional 8 rabbits per group after unilateral implantation of each of the 3 carriers with 0.1 mg/mL rhBMP-7. Torsional strength of treated radii at 6 weeks were normalized against contralateral radii and reported as % of normal strength. Additionally, serum was collected at baseline and weekly until endpoint for anti-rhBMP-7 ADA response, as well as systemic drug levels (PK). Primary outcome measurements were radiographic healing, biomechanical testing, and preliminary immunogenicity testing results.

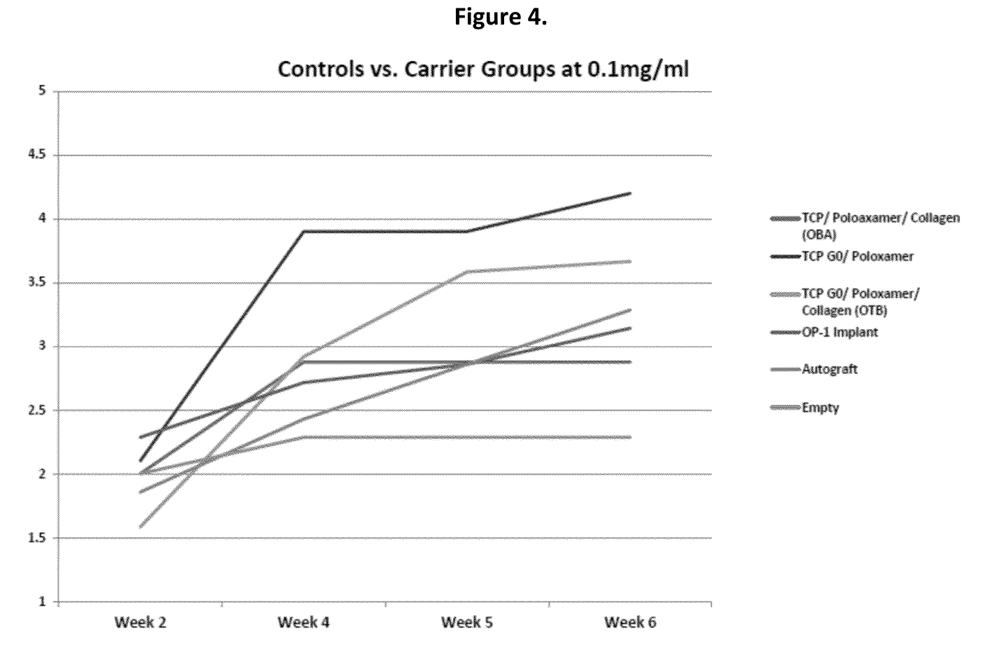

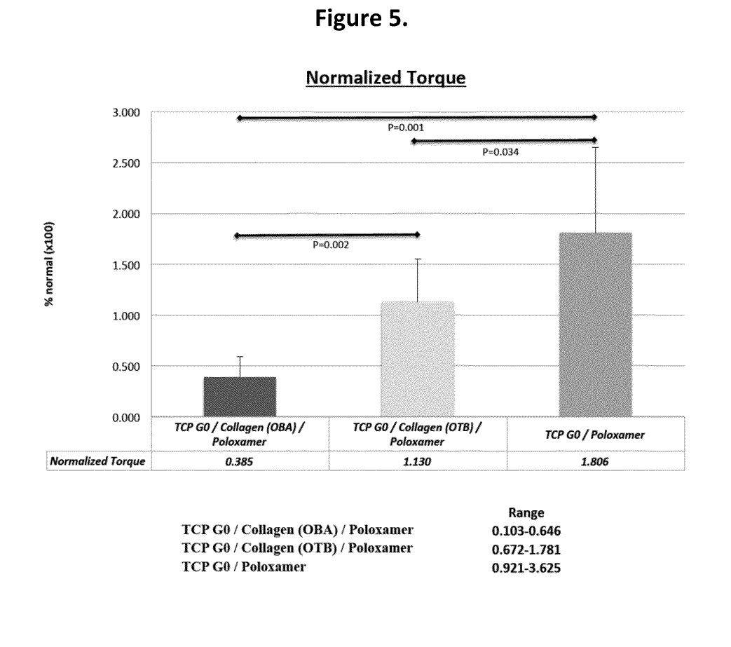

As show in FIG. 4, the overall radiographic healing response was greatest in the rhBMP-7 containing TCP/poloxamer carrier, followed by the same carrier combined with atelocollagen (described as OTB collagen in FIG. 4), and lastly the same carrier combined with telocollagen (described as OBA collagen in FIG. 4). The improved healing response in the TCP/poloxamer carrier with rhBMP-7 was sustained throughout the duration of the study at all dose levels and demonstrated improvement over autograft and OP-1 Implant. Radiographic healing in TCP/poloxamer was optimal at 0.1 mg/mL rhBMP-7 and plateaued or decreased slightly at 1.0 mg/mL, suggesting 0.1 mg/mL rhBMP-7 is the minimally efficacy dose with the therapeutic dose range of 0.1-1.0 mg/mL from the doses tested. The peak level of healing induced by TCP/poloxamer with 0.1 mg/mL rhBMP-7 was represented by a mean radiographic score of 4.2, suggesting bridging across the defect with new bone as well as initiation of remodeling (for reference, a score of 3.5 or higher suggests initiation of union). The superior radiographic healing results of TCP/poloxamer over other carriers and over autograft and OP-1 Implant was unexpected. The improvement over autograft without the need to harvest in a second site is a clear advantage of the TCP/poloxamer. Moreover, the TCP/poloxamer with rhBMP-7 was more efficacious at a lower dose level compared to the currently-marketed OP-1 Implant, and exhibited improved safety with reduced immunogenicity. The biomechanical strength (torsional strength) of the treated radius in the TCP/poloxamer with 0.1 mg/mL rhBMP-7 was 181% of the strength of the intact radius, compared with 113% for the atelocollagen enhanced material containing 0.1 mg/mL rhBMP-7 and only 39% for the telocollagen enhanced material containing 0.1 mg/mL rhBMP-7 (FIG. 5). These differences in mechanical strength were statistically significant when compared by t-test.

### Example 8

### Reduced Immunogenicity to rhBMP-7

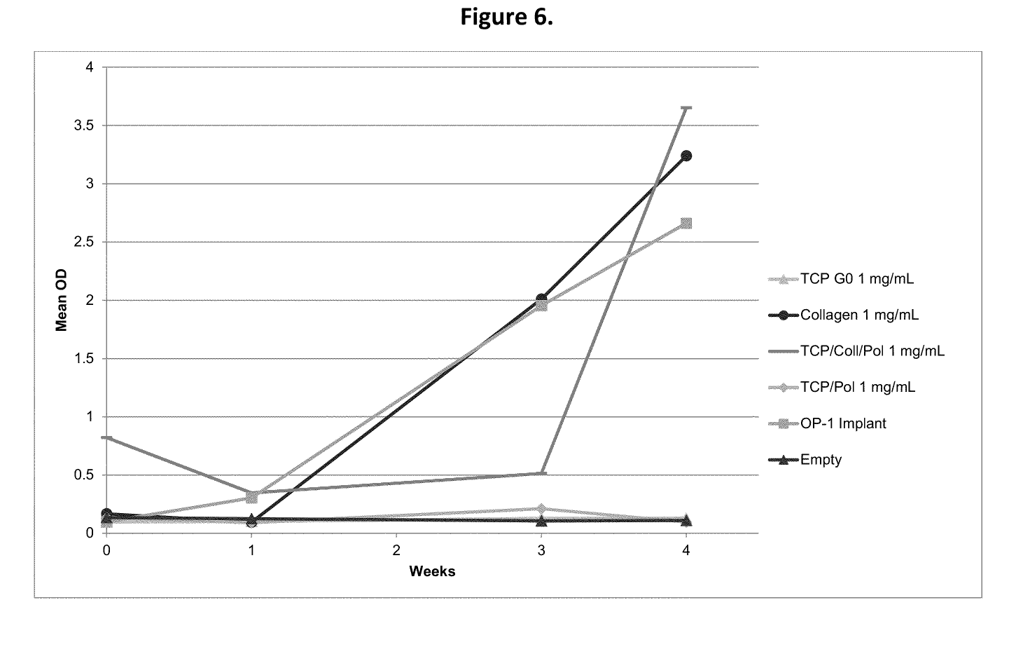

The antibody response against rhBMP-7 in vivo was also investigated. In the rabbit femoral condyle bone defect study, local inflammatory response was mostly mild in the TCP/poloxamer carrier with rhBMP-7, compared to higher level of inflammation in the telocollagen carrier with rhBMP-7 or OP-1 Implant. Without wishing to be bound to any theory, the decrease in inflammation in the TCP/poloxamer treated bones is thought to be related to the lower level of bone formation observed for collagen-containing carriers, as inflammation may interfere with the in-migration and differentiation of pluripotent cells capable of differentiating into the osteoblastic lineage. Preliminary immunogenicity differences were also observed between rhBMP-7 delivered with either telocollagen or TCP/poloxamer (FIG. 6). The anti-drug-antibody (ADA) response against rhBMP-7 was examined in serum from animals treated with TCP/poloxamer-containing compositions of the invention or currently available collagen-based carriers. All collagen carrier serum samples (n=4) at weeks 3 and 4 (and not at any other time points) screened ADA positive, as did all OP-1 Implant serum samples at weeks 3 and 4 (n=2). In contrast, ADA response against rhBMP-7 was screened negative in all TCP/poloxamer serum samples tested (n=2). Negative screening results were also seen in rhBMP-7 combined with TCP alone (without poloxamer) (n=8).

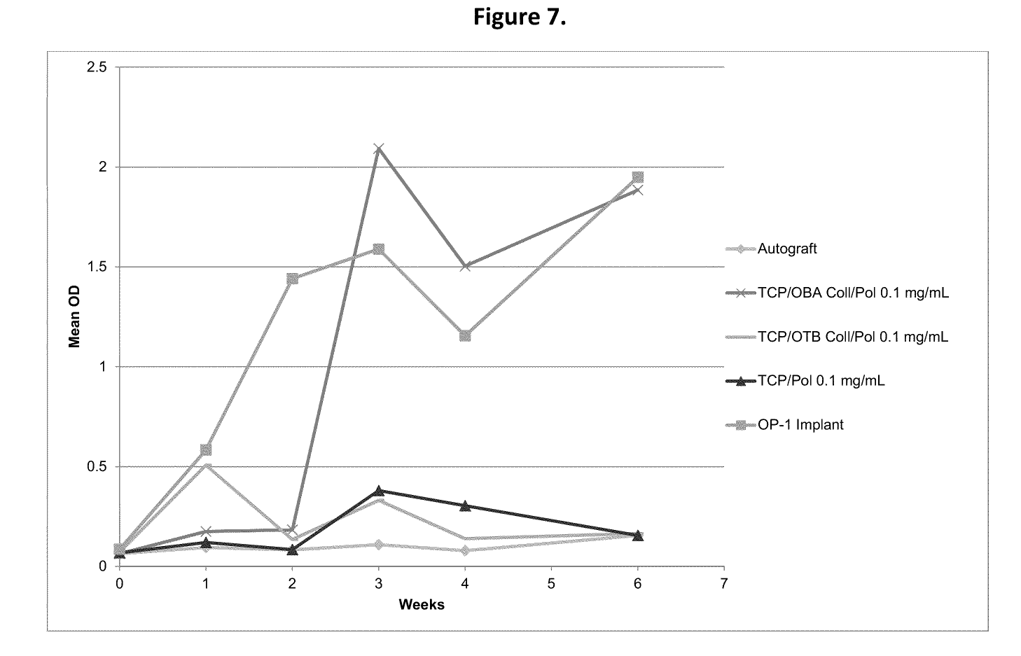

ADA assay results from the rabbit segmental defect study confirmed a trend observed in the previous rabbit femoral condyle data in which differential ADA results were seen in implants comprising TCP/poloxamer or TCP/poloxamer with telocollagen. An ADA response against rhBMP-7 initiated during weeks 1-2 was observed in all telocollagen containing carrier serum samples (n=5 for 0.1 mg/mL and n=6 for 1.0 mg/mL rhBMP-7) by week 6 and comparable to ADA positive results in 6/7 OP-1 Implant serum samples (FIG. 7). The level of positive screening response observed in the telocollagen containing carrier samples were rhBMP-7 dose dependent, suggesting potential higher titer levels of antibody response with increased rhBMP-7 concentration. In contrast, little to no ADA response against rhBMP-7 was observed in the TCP/poloxamer serum samples (4/4 and 3/4 for 0.1 mg/mL and 1.0 mg/mL rhBMP-7, respectively) throughout the study.

Interestingly, no ADA response against rhBMP-7 was found in atelocollagen containing TCP/poloxamer serum samples (6/6 and 5/5 for 0.1 mg/mL and 1.0 mg/mL rhBMP-7, respectively) throughout the study, suggesting that telocollagen may be a factor in the generation of the anti-rhBMP-7 response by possibly acting as an adjuvant to elicit rhBMP-7 immunogenicity. All samples tested positive were subsequently re-tested and confirmed positive.

### Example 9

### Disintegration and Degradation of Carrier

Osteogenic implants containing carriers and osteogenic proteins according to the invention exhibit superior performance and safety relative to currently available products. In the preferred embodiment of β-TCP/poloxamer carrier delivered with rhBMP-7, for example, the temporary binding effects of the poloxamer binder component contribute to favorable handling properties including moldability prior to implantation, then disintegrates over time in vivo by dissolution of the poloxamer component, leaving the β-TCP granules at the site of implantation.

The β-TCP used in the current invention (Ca₃(PO₄)₂) has a calcium to phosphorous ratio of 1.5, and preferably degrades on a timescale that matches up well with the rate of new bone formation. β-TCP alone would be expected to degrade in vivo in the 12-24 week time frame; however, bone remodeling promoted by the osteoinductive agent would likely accelerate the degradation of TCP. In preferred embodiments, the rate at which β-TCP within the implant degrades should approximate the rate at which new bone is formed. Complete healing of orthopedic injuries in patients treated with osteoinductive proteins such as BMPs generally takes about 6-24 weeks, and ideally about 8-12 weeks. Therefore, in preferred embodiments, the rate of degradation of the implant is 6-24 weeks, with 8-12 weeks being more preferable.

### Example 10

### Method of Use and Delivery

Osteogenic implants of the invention generally take the form of a moldable putty or injectable paste, and are preferably delivered by implantation, extrusion, or injection either through open reduction surgery or percutaneous injection into skeletal sites that may require repair, regeneration, augmentation, or grafting. Implants of the invention can be used for a variety of orthopedic indications including long bone nonunions, fracture repair, spine fusion, foot/ankle fusion, vertebral compression fractures, local bone augmentation, oral/maxillofacial surgery, osteoporosis, osteolysis, and other bone defect/injury repair.

The invention also includes kits comprising carriers of the invention and osteoinductive proteins for use in treating patients. In preferred embodiments, the carrier component is prefilled in a vial or a syringe and sterilized by gamma-irradiation. The osteogenic protein component is provided separately in another vial where it has been aseptically filled and lyophilized (without gamma irradiation). The aseptically filled and lyophilized protein, preferably rhBMP-7, is reconstituted with water for injection (WFI) or saline prior to use, and is then mixed with the carrier component prior to implantation or injection. In some embodiments, however, the carrier and osteogenic protein components are prepared aseptically as a unitary device in a vial or syringe and co-lyophilized. This unit can be subsequently reconstituted with fluid to form the putty or paste prior to implantation or injection. In other embodiments, the reconstituted putty or paste is prefilled in a vial or syringe by aseptic processing and provided in ready to use format. The preferred embodiment comprises the carrier component in a pre-filled syringe that has been gamma sterilized, to be combined with a reconstituted osteogenic protein in a vial that has been aseptically filled and lyophilized. In the preferred embodiment, the pre-filled syringe is also a mixing syringe with an actuator so that the carrier hydrated with the reconstituted osteogenic protein can be mixed inside the syringe by manual rotation and actuation without exposure of the sterile contents in open air, thus minimizing the risk of contamination while improving the mixing process. Once mixed, the implant can be extruded through a cannula or large bore needle directly onto the treatment site, or it can be extruded into a surgeon's hand and further molded into a desired shape prior to implantation. The force needed to extrude or inject from the syringe is typically less than 100N. The carrier and osteogenic protein components may be prepared in ambient temperature and pressure conditions.

Taken together, the results of these studies indicate that implants and carriers of the present invention have the potential to promote robust bone growth at a level greater than currently marketed BMP-containing products while minimizing the risks of inflammation and ADA response that are sometimes seen in these products.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “protein” means any molecule or collection of molecules that includes a substantial amino acid component, including without limitation, protein aggregates, multi-subunit proteins, protein fragments, cleavage products, protein conjugates, protein fractions, polypeptides, glycopeptides, protein complexes, and the like. The term “consists essentially of” means excluding other materials that contribute to function, unless otherwise defined herein. Nonetheless, such other materials may be present, collectively or individually, in trace amounts.

As used in this specification, the terms “substantially,” “approximately” or “about” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have been described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A concentrate dilutable to form a therapeutic material, the concentrate comprising:
between about 50% and about 80% by mass of β-tricalcium phosphate in particulate form; and
between about 20% and about 50% by mass of a copolymer of polyethylene oxide and polypropylene oxide having a molecular weight range of between about 6,840 and about 17,400.

2. The concentrate of claim 1, wherein the copolymer has a molecular weight range of between about 9,840 and about 14,600.

3. The concentrate of claim 2, wherein the copolymer is poloxamer 407.

4. The concentrate of claim 1, further comprising a collagen.

5. The concentrate of claim 4, wherein the collagen is atelocollagen.

6. The concentrate of claim 4, wherein the collagen is in particulate form.

7. The concentrate of claim 1, wherein a particle of β-tricalcium phosphate has a diameter of between about 0.1 mm and about 3.0 mm.

8. The concentrate of claim 7, wherein the β-tricalcium phosphate has a diameter of between about 0.25 mm and about 1.5 mm.

9. The concentrate of claim 8, wherein the β-tricalcium phosphate has a diameter of between about 0.25 mm and about 0.5 mm.

10. The concentrate of claim 1, wherein a particle of β-tricalcium phosphate (a) has a porosity of between about 50% and about 90%, and (b) includes a micropore and a macropore.

11. The concentrate of claim 10, wherein a particle of β-tricalcium phosphate (a) has a porosity of about 75%, and (b) includes a micropore and a macropore.

12. The concentrate of claim 1, wherein the β-tricalcium phosphate particles include hydroxyapatite.

13. A kit for treating a patient, comprising:
a concentrate, comprising:
β-tricalcium phosphate in particulate form; and
a copolymer of polyethylene oxide and polypropylene oxide having a molecular weight range of between about 6,840 and about 17,400.

14. The kit of claim 13, further comprising:
a bone morphogenetic protein; and
an aqueous diluent.

15. The kit of claim 14, further comprising an instruction set setting forth a method comprising the steps of:
adding the diluent to the lyophilized bone morphogenetic protein to form a bone morphogenetic protein solution; and
mixing a first quantity of the bone morphogenetic protein solution with a second quantity of the concentrate to form a therapeutic material.

16. The kit of claim 15, wherein the copolymer is poloxamer 407.

17. The kit of claim 15, wherein the therapeutic material is flowable, and the method further comprises the step of applying the therapeutic material to a body of a patient.

18. The kit of claim 15, wherein the therapeutic material is moldable, and the method further comprises the step of molding the therapeutic material to at least partially fill a void within a bone of a patient.

19. The kit of claim 14, wherein said bone morphogenetic protein is rhBMP-7.

20. The kit of claim 14, further comprising a syringe in which said concentrate and said bone morphogenetic protein are mixed.

21. The kit of claim 13, wherein the copolymer has a molecular weight of between about 9,840 and about 14,600.
